(19) United States
(12) Patent Application Publication
(10) Pub. No.: US 2026/0083423 A1
(43) Pub. Date: Mar. 26, 2026

(54) METHOD FOR CONTROLLING AN X-RAY DEVICE AND X-RAY DEVICE

(71) Applicant: Siemens Healthineers AG, Forchheim (DE)

(72) Inventor: Alois Regensburger, Poxdorf (DE)

(21) Appl. No.: 19/340,842

(22) Filed: Sep. 25, 2025

(30) Foreign Application Priority Data
Sep. 26, 2024 (DE) 10 2024 209 339.4

(51) Int. Cl. A61B 6/00; A61B 6/03; A61B 6/08; A61B 6/58

(52) U.S. Cl. CPC A61B 6/547; A61B 6/035; A61B 6/08; A61B 6/584

(57) ABSTRACT

A method for controlling an X-ray device including an X-ray source and an X-ray detector arranged opposite one another and in a first defined arrangement is provided. A light guiding device is arranged in a second defined arrangement. Planning information is received for arranging a medical object, and a light fan is emitted using the light guiding device, such that a virtual reference beam is arranged within the light fan. The first defined arrangement is repositioned from an initial position to a further position. The virtual reference beam intersects a planned path in the initial position and further position. The further position is captured as the current position. The second defined arrangement is rotated based on the planning information and the current position, such that the light fan illuminates the planned path.

This application claims the benefit of German Patent Application No. DE 10 2024 209 339.4, filed on Sep. 26, 2024, which is hereby incorporated by reference in its entirety.

### BACKGROUND

The present embodiments relate to controlling an X-ray device.

In medical interventions, precise placement of medical objects (e.g., a needle and/or a medical instrument) is often crucial (e.g., in minimally invasive procedures, such as biopsies for pain therapy and/or catheter placements). Herein, it is often necessary to move the medical object along a planned trajectory to a target location and/or for the medical object to be aligned along a planned trajectory (e.g., a planned spatial direction).

Needles may, for example, be guided on medical C-arm X-ray devices and/or a computed tomography system (CT system) using optical and/or electromagnetic tracking of the needle and/or laser needle guidance. Systems for optical and/or electromagnetic tracking may be expensive and complex to use: such systems may be prone to malfunction, require regular calibration, and/or their accuracy may be impaired by external influences such as metallic objects and electromagnetic fields.

In the field of laser needle guidance on C-arm X-ray devices, for example, two “progression views” of the C-arm X-ray device may be used; these are directed as perpendicularly as possible to a planned needle path and are generally perpendicular to a “bulls-eye” direction (e.g., a longitudinal extension direction of the planned needle path). These may be calculated in advance according to specific geometric criteria and to avoid collisions. In order to move to one of the progression views, the one progression view may have to be selected in the system menu to enable the C-arm X-ray device to automatically move to this position. The line laser may then be switched on, and the needle path may be illuminated from the side. Herein, a line laser integrated in a detector of the C-arm X-ray device may enable its lateral progression view in which the planned needle path may be illuminated from the side. Selecting the progression view in the system menu may be time-consuming and cumbersome. The precalculated progression views also limit flexibility and efficiency.

These disadvantages lead to more difficult and less efficient guidance of medical objects under X-ray imaging control.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, improved guidance of medical objects under X-ray imaging control is enabled.

Independent of the grammatical term usage, individuals with male, female, or other gender identities are included within the term.

In a first aspect, the present embodiments relate to a method for controlling an X-ray device. The X-ray device includes an X-ray source, an X-ray detector, and a light guiding facility. The X-ray source and the X-ray detector are arranged opposite one another and in a first defined arrangement. The first defined arrangement is movably mounted. The light guiding facility is arranged in a second defined arrangement with respect to the X-ray detector. The second defined arrangement is at least rotatably mounted. Further, a virtual reference beam is defined through a first reference point and a second reference point. Herein, the first reference point is arranged at the X-ray source, and the second reference point is arranged between the X-ray source and the X-ray detector as part of the first defined arrangement. The method includes a plurality of acts. In a first act, planning information is received on a planned path for arranging a medical object that may be mapped using the medical X-ray device. In a further act, a light fan is emitted by the light guiding unit (e.g., light guiding device) such that the virtual reference beam is arranged within the light fan. In a further act, the first defined arrangement is repositioned from an initial position to a further position. The virtual reference beam intersects the planned path in the initial position and the further position of the first defined arrangement (e.g., at an intersection point in each case). The intersection points of the virtual reference beam with the planned path in the initial position and further positions of the first defined arrangement may be the same or different. In a further act, the further position is captured as the current position of the first defined arrangement. In a further act, the second defined arrangement is rotated based on the planning information and the current position of the first defined arrangement, such that the light fan illuminates the planned path.

The X-ray device may, for example, be a medical X-ray device. The X-ray source may be configured to emit X-rays (e.g., an X-ray beam bundle). Further, the X-ray detector may be configured to capture (e.g., detect) incident X-rays (e.g., at an X-ray sensitive surface). The X-ray detector may, for example, be configured as a flat detector.

The X-ray source and the X-ray detector are arranged opposite one another. For example, the X-ray source and the X-ray detector are arranged opposite one another such that the X-rays that may be emitted by the X-ray source illuminate the X-ray detector (e.g., the X-ray sensitive surface of the X-ray detector). Further, the X-ray source and the X-ray detector are arranged in the first defined arrangement. Herein, the first defined arrangement may characterize a relative position (e.g., a relative spatial position and/or relative alignment and/or relative pose) between the X-ray source and the X-ray detector. Further, the X-ray source and the X-ray detector may be arranged on a common holding structure (e.g., a C-arm and/or the like and/or O-arm) in the first defined arrangement. The common holding structure may be movably mounted about one or more axes.

The first defined arrangement is movable (e.g., in a translatable and/or rotatable manner). For example, the X-ray source and the X-ray detector may be movably mounted in the first defined arrangement (e.g., in a translatable and/or rotatable manner).

The light guiding facility is arranged in the second defined arrangement with respect to the X-ray detector. Herein, the second defined arrangement may characterize a relative position (e.g., a relative spatial position and/or relative alignment and/or relative pose) between the light guiding facility and the X-ray detector. The second defined arrangement is mounted at least rotatably (e.g., in a translatable and rotatable manner). For example, the light guiding facility and the X-ray detector in the second defined arrangement may be mounted at least rotatably (e.g., in a translatable and rotatable manner). When the X-ray detector is configured as a flat detector, an axis of rotation of the second defined arrangement may correspond to a surface normal of the flat detector.

The light guiding facility may include a light source (e.g., a laser light source) that is configured to emit the light fan. For this purpose, the light guiding facility may, for example, include an optical diaphragm. The light fan may illuminate a predefined layer (e.g., in a fan shape). In one embodiment, the light guiding facility may be arranged with respect to the X-ray detector such that the light fan may be emitted at least partially in the direction of the X-ray source. For example, at least in a subset of possible positions of the first and second defined arrangements (e.g., in all possible positions of the first and second defined arrangement), the light guiding facility may be arranged with respect to the X-ray detector such that the light fan may be emitted at least partially in the direction of the X-ray source. The light guiding facility may emit the light fan as a laser line, for example.

When the first defined arrangement is moved, the second defined arrangement may be moved along with it. A movement of the second defined arrangement (e.g., at least rotation of the second defined arrangement) may take place independently of the first defined arrangement (e.g., with respect to the first defined arrangement).

The reception of the planning information may, for example, include capturing and/or reading a computer-readable data memory and/or reception from a data storage unit (e.g., a database). Further, the planning information may be provided by a provisioning unit of a medical imaging device.

The planning information may include spatially (e.g., spatially and temporally) resolved information on a planned path for arranging the medical object. Herein, the planned path may run in a straight line, at least in sections (e.g., completely). For example, the planning information may be provided based on a pre-procedural data set (e.g., a pre-procedural 3D data set) of an examination object.

The medical object may be a surgical instrument (e.g., a needle, such as a puncture needle, and/or a drill) and/or a diagnostic instrument (e.g., an endoscope, such as a laparoscope), and/or a catheter, and/or an implant. In one embodiment, the medical object may at least partially (e.g., completely) be configured as rigid and elongated (e.g., rod-shaped and/or needle-shaped). In one embodiment, the medical object may be mapped by the medical object (e.g., may be configured as at least partially X-ray opaque). In one embodiment, the planning information may specify the planned path with respect to the examination object (e.g., with respect to a coordinate system of the examination object). The examination object may, for example, be a human and/or veterinary patient and/or an examination phantom.

If the medical object includes a needle, the planned path may, for example, include a planned needle trajectory.

The first reference point may include a spatial point (e.g., determined by spatial coordinates) that is arranged at the X-ray source (e.g., a focal point of the X-ray source). The first reference point may include a defined (e.g., constant) positional relationship to the X-ray source.

The second reference point may include a spatial point (e.g., determined by spatial coordinates) that is different than the first reference point and is arranged between the X-ray source and the X-ray detector. The second reference point may be arranged on the X-ray detector, at the X-ray source or in an intermediate region between the X-ray source and the X-ray detector. The second reference point may be spaced apart from the first reference point. The first reference point and the second reference point are part of the first defined arrangement.

In one embodiment, the first reference point and the second reference point may define the virtual reference beam. The virtual reference beam may run from the first reference point through the second reference point.

In a further act, the light guiding unit emits the light fan such that the virtual reference beam is arranged within the light fan. Herein, the light fan may have a defined positional relationship with respect to the second defined arrangement (e.g., with respect to the light guiding facility and the X-ray detector). This further allows the light fan to have a defined positional relationship with respect to the first defined arrangement. The light fan may illuminate a predefined layer (e.g., in a fan shape). In one embodiment, the light guiding unit emits the light fan such that the virtual reference beam is arranged within the layer illuminated by the light fan.

In a further act, the first defined arrangement may be repositioned (e.g., moved) from an initial position to a further position (e.g., manually or semi-automatically). For example, a user may be supported in the semi-automatic repositioning of the first defined arrangement by digital suggestions (e.g., a workflow tip). The initial position of the first defined arrangement may describe an initial spatial position and/or alignment and/or pose of the first defined arrangement at a first point in time. Further, the further position may describe a further spatial position and/or alignment and/or pose of the first defined arrangement at a further point in time (e.g., after the first point in time). The further position is different from the initial position of the first defined arrangement. Further, the virtual reference beam intersects the planned path (e.g., in each case at an intersection point) when the first defined arrangement is arranged in the initial position and further position. The repositioning of the first defined arrangement may include translation and/or rotation of the first defined arrangement. For example, the repositioning does not only include rotation of the first defined arrangement about the planned path as the axis of rotation.

In one embodiment, the second defined arrangement may be positioned with respect to the first defined arrangement at the initial point in time such that the light fan illuminates the planned path. After repositioning of the first defined arrangement from the initial position to the further position, the second defined arrangement may be positioned with respect to the planned path such that the light fan only intersects the planned path (e.g., initially). For example, the intersection point of the virtual reference beam with the planned path may be arranged at a center of rotation (e.g., an isocenter) of the first defined arrangement.

In a further act, the further positioning of the first defined arrangement is captured as the current position of the first defined arrangement. The current position of the first defined arrangement may be captured by a control unit for controlling a movement (e.g., the repositioning) of the X-ray device (e.g., of the first defined arrangement). For example, the control unit may have a sensor (e.g., an electromagnetic and/or optical and/or acoustic and/or mechanical sensor) for capturing the current position of the first defined arrangement. In one embodiment, the current position of the first defined arrangement may be captured with respect to the planned path (e.g., with respect to a coordinate system of the examination object).

In a further act, the second defined arrangement is rotated based on the planning information (e.g., the information on the planned path) and the current position of the first defined arrangement such that the light fan illuminates the planned path. For example, the rotation of the second defined arrangement enables the light fan to be rotated with respect to the planned path. The rotation of the second defined arrangement may take place simultaneously or after the repositioning of the first defined arrangement.

In one embodiment, the current position of the first defined arrangement may be used to determine the current position of the second defined arrangement (e.g., with respect to the planned path). Based on the planning information (e.g., the information on the spatial position of the planned path) and the current position of the first defined arrangement (e.g., the current position of the second defined arrangement), the second defined arrangement (e.g., the light fan) may be rotated such that the light fan illuminates the planned path.

In one embodiment, this makes it possible for the light fan to always illuminate the planned path for arranging the medical object. This may enable flexible and precise guidance of the medical object along the planned path. Rotation of the second defined arrangement (e.g., the X-ray detector) with respect to the planned path (e.g., a needle path) may enable collimation along the planned path; this not only helps to reduce the X-ray dose, but also provides regions of interest of the examination object to be mapped may still be mapped.

In a further embodiment of the method, the second reference point may be arranged at a center of rotation (e.g., an isocenter) of the first defined arrangement, at a center of rotation of the second defined arrangement, or at a geometric center of a beam exit window of the X-ray source.

The center of rotation (e.g., a center of rotation) of the first defined arrangement may designate a point in space about which the first defined arrangement is rotatably mounted. For example, an axis of rotation for rotation of the first defined arrangement may run through the center of rotation. For example, the center of rotation of the first defined arrangement may be an isocenter of the X-ray device. In one embodiment, the second reference point may be arranged at the center of rotation of the first defined arrangement. This allows the virtual reference beam to be rotated about the second reference point upon rotation of the first defined arrangement.

Alternatively, the second reference point may be arranged at a center of rotation (e.g., a center of rotation) of the second defined arrangement. The center of rotation (e.g., the center of rotation) of the second defined arrangement may designate a point in space about which the second defined arrangement is rotatably mounted. Herein, an axis of rotation of the second defined arrangement may run through the center of rotation of the second defined arrangement. In one embodiment, the axis of rotation of the second defined arrangement may be arranged perpendicular to a surface of the X-ray detector facing the X-ray source. The center of rotation of the second defined arrangement may, for example, be a center of rotation of the X-ray detector. For example, the center of rotation of the second defined arrangement may be arranged at a geometric center of the X-ray detector (e.g., the X-ray sensitive surface of the X-ray detector). This allows the second reference point to be invariant with respect to rotation of the second defined arrangement. Herein, the virtual reference beam may strike the same point in space (e.g., a detector pixel and/or a location on the X-ray detector) even upon rotation of the second defined arrangement.

Alternatively, the second reference point may be arranged at a geometric center of the beam exit window of the X-ray source. Herein, the virtual reference beam may run along a center beam and/or middle beam of an X-ray beam bundle that may be emitted by the X-ray source. This allows the reference beam to be invariant with respect to a change in the relative position of the X-ray source and the X-ray detector in the first defined arrangement (e.g., due to mechanical deformation of the common holding structure). The emission of the light fan by the light guiding facility may be adapted accordingly upon identification of a change in the relative position.

In a further embodiment of the method, the virtual reference beam may be arranged along an axis of rotation of the second defined arrangement.

In one embodiment, the second reference point may be arranged along the axis of rotation of the second defined arrangement (e.g., on the axis of rotation of the second defined arrangement). Herein, the axis of rotation of the second defined arrangement may, for example, run through the center of rotation of the first defined arrangement and/or the geometric center of the beam exit window. Alternatively, the second reference point may be arranged at the center of rotation of the second defined arrangement.

This allows the virtual reference beam to be invariant with respect to rotation of the second defined arrangement.

In a further embodiment of the method, the repositioning of the first defined arrangement may be limited to rotation about an intersection point of the virtual reference beam with the planned path or a combination including rotation about the intersection point and translation parallel to the planned path.

In one embodiment, the repositioning (e.g., the degrees of freedom of movement of the repositioning) of the first defined arrangement may be limited to the rotation (e.g., rotational movement) of the first defined arrangement about the intersection point of the virtual reference beam with the planned path. Alternatively, the repositioning (e.g., the degrees of freedom of movement of the repositioning) of the first defined arrangement may be limited to the rotation (e.g., the rotational movement) of the first defined arrangement about the intersection point of the virtual reference beam with the planned path and a translation (e.g., translational movement) of the first defined arrangement parallel to the planned path. By limiting translation (e.g., the translational movement) of the first defined arrangement parallel to the planned path, it may be ensured that the virtual reference beam intersects the planned path at an intersection point. Limiting the repositioning (e.g., of the degrees of freedom of movement) of the first defined arrangement may, for example, include adapting a control (e.g., adapting degrees of freedom of control) of the first defined arrangement. For example, control elements and the like may be assigned via an input unit (e.g., a user interface) of the X-ray device such that the first defined arrangement may only be moved with a geometric limitation; for example, joysticks of the input unit may be adjusted accordingly in their function.

For example, the intersection point of the virtual reference beam with the planned path may be arranged at a center of rotation (e.g., an isocenter) of the first defined arrangement. Herein, the first defined arrangement may be repositioned from the initial position to the further position (e.g., manually or semi-automatically; rotated isocentrically) such that the second defined arrangement is in each case rotated automatically such that the light fan illuminates the planned path (e.g., in progression view).

This may provide that the virtual reference beam intersects the planned path even during repositioning of the first defined arrangement from the initial position to the further position (e.g., at an intersection point). This may enable continuous repositioning of the first defined arrangement while maintaining the intersection point between the virtual reference beam and the planned path. Further, this may provide that the light fan illuminates the planned path (e.g., continuously) at least partially.

In a further embodiment of the method, a change in the relative position of the first and second reference points may be identified. The emission of the light fan by the light guiding facility may be adapted based on the identified change in the relative position.

The relative position of the X-ray source and the X-ray detector in the first defined arrangement may change (e.g., due to mechanical deformation of the common holding structure). For example, the relative position of the X-ray source and the X-ray detector may change in dependence on the current position of the first defined arrangement. Herein, this may result in a change in the relative position of the first and second reference points. The change in the relative position of the first and second reference points may result in a change in the relative position of the virtual reference beam with respect to the second defined arrangement (e.g., of the emittable light fan). In one embodiment, the change in the relative position of the first and second reference points may be identified (e.g., automatically). Identifying the change in the relative position of the first and second reference points may, for example, include capturing the current relative position of the X-ray source and the X-ray detector (e.g., by a sensor). Alternatively or additionally, the change in the relative position of the first and second reference points may be identified based on the current position of the first defined arrangement (e.g., based on a look-up table and/or a physical model of the X-ray device).

In one embodiment, the light guiding facility may be configured to adapt the emission of the light fan (e.g., a projection direction and/or a fan angle of the light fan) in dependence on the identified change in the relative position of the first and second reference points. In one embodiment, the light guiding facility may adapt the emission of the light fan in dependence on the identified change in the relative position such that the virtual reference beam is arranged within the light fan (e.g., within the layer illuminated by the light fan).

The embodiment may enable compensation of a change in the relative position of the first and second reference points during the emission of the light fan. This may provide that the virtual reference beam is arranged within the light fan.

In a further embodiment of the method, the first defined arrangement may be repositioned into a plurality of further positions (e.g., continuously). Herein, the virtual reference beam may intersect the planned path in the initial position and further positions of the first defined arrangement. The respective current position of the first defined arrangement may be captured. The second defined arrangement may be rotated based on the planning information and the respective current position of the first defined arrangement such that the light fan illuminates the planned path.

In one embodiment, the first defined arrangement may be repositioned (e.g., moved) into a number of (e.g., several) further positions in a temporal sequence. The plurality of further positions may be at least partially (e.g., completely) different. In one embodiment, the initial position and the plurality of further positions may describe a trajectory (e.g., a continuous trajectory) of the first defined arrangement. In one embodiment, the first defined arrangement may be repositioned continuously from the initial position to the plurality of further positions (e.g., along a continuous trajectory in temporal sequence).

The virtual reference beam may intersect the planned path in the initial position and further positions of the first defined arrangement (e.g., at an intersection point in each case). The intersection points of the virtual reference beam with the planned path in the initial position and further positions of the first defined arrangement may be at least partially (e.g., completely) the same or different.

Herein, the respective current position of the first defined arrangement may be captured each time the first defined arrangement is arranged in one further position of the plurality of further positions.

In one embodiment, the second defined arrangement may be rotated based on the planning information (e.g., the information on the position of the planned path) and the respective current position of the first defined arrangement such that the light fan illuminates the planned path (e.g., such that the planned path is arranged within the layer illuminated by the light fan). The rotation of the second defined arrangement may take place simultaneously or after the repositioning of the first defined arrangement.

The embodiment may enable particularly flexible and efficient positioning of the first defined arrangement. Rotation of the second defined arrangement provides illumination (e.g., continuous illumination) of the planned path by the light fan.

In a further embodiment of the method, the medical object may be arranged along the planned path. X-rays for illuminating the medical object may be emitted by the X-ray source. The X-rays may be captured by the X-ray detector, and a signal may be provided in dependence on the captured X-rays. X-ray image data may be provided in dependence on the signal.

In one embodiment, the medical object (e.g., a longitudinal axis of extension of the medical object) may be arranged along the planned path (e.g., on the planned path). In one embodiment, the X-ray source may emit X-rays (e.g., an X-ray beam bundle) for the illumination of the medical object. The X-rays may be captured (e.g., detected) by the X-ray detector (e.g., the X-ray sensitive surface of the X-ray detector, such as after interaction with the medical object). The X-ray detector may provide a signal in dependence on the captured X-rays. In one embodiment, the signal may include information (e.g., spatially or spatio-temporally resolved information) on the captured X-rays. The signal may be provided by the X-ray detector. In one embodiment, the X-ray image data may be provided in dependence on the signal.

The X-ray image data may include a representation (e.g., mapping) of the medical object (e.g., of the medical object and the examination object). Herein, the X-ray image data may be spatially resolved in two dimensions (2D) and/or three dimensions (3D). In addition, the X-ray image data may be time-resolved. The X-ray image data may have a plurality of image points (e.g., pixels and/or voxels) in each case with at least one image value (e.g., a plurality of image values, such as time-intensity curves) that in each case map a partial volume.

The provision of the X-ray image data may include storage on a computer-readable storage medium (e.g., a non-transitory computer-readable storage medium) and/or a display on a representation unit and/or transmission to a representation unit. For example, a graphical representation of the X-ray image data may be displayed by the representation unit (e.g., a display).

The embodiment may enable improved guidance of medical objects under X-ray imaging control.

According to a further embodiment of the method, mapping of the medical object may be identified in the X-ray image data (e.g., manually or automatically, such as by segmentation and/or pattern recognition and/or annotation). Herein, a longitudinal axis of extension (e.g., the current longitudinal axis of extension) of the medical object may be identified based on the identified mapping of the medical object in the X-ray image data. Herein, the current alignment of the medical object and its virtual continuation may be adopted as the planned path.

In this embodiment, the light fan may illuminate the current longitudinal extension direction of the medical object as the planned path after rotation of the second defined arrangement. This may assist medical personnel (e.g., when positioning and/or advancing the medical object) in maintaining the current alignment of the medical object.

In a further embodiment of the method, the planning information may be registered with a coordinate system of the X-ray device.

Registering the planning information with the coordinate system of the X-ray device may include applying a transformation rule to the planning information. Herein, the transformation rule may specify translation and/or rotation and/or scaling and/or deformation that minimizes any deviation between corresponding spatial points of the planning information (e.g., a coordinate system of the planning information and the coordinate system of the X-ray device). If the planning information is registered with a coordinate system of the examination object, the coordinate systems of the examination object and the X-ray device may be registered with one another.

The embodiment may enable particularly precise guidance of medical objects under X-ray imaging control.

In a further embodiment of the method, the provision of the X-ray image data may include the provision of a graphical representation of the X-ray image data by a representation unit. Herein, the graphical representation of the X-ray image data may be rotated such that the current rotation of the second defined arrangement with respect to the planned path is compensated.

The representation unit may, for example, include a monitor and/or a display and/or a projector that is configured to display the graphical representation of the X-ray image data. The provision of the graphical representation of the X-ray image data may include displaying the graphical representation of the X-ray image data using the representation unit. The graphical representation of the X-ray image data may be rotated (e.g., digitally). Herein, the graphical representation of the X-ray image data may be dependent on the current position (e.g., the current rotation) of the second defined arrangement with respect to the planned path. This may enable consistent representation of the mapping of the medical object (e.g., independently of the current rotation of the second defined arrangement).

In a second aspect, the present embodiments relate to an X-ray device including an X-ray source, an X-ray detector, a light guiding facility (e.g., a light guiding device), and a control unit (e.g., a controller). The X-ray source and the X-ray detector are arranged opposite one another and in a first defined arrangement. The first defined arrangement is movably mounted. The light guiding facility is arranged in a second defined arrangement with respect to the X-ray detector. The second defined arrangement is at least rotatably mounted. Herein, a virtual reference beam is defined by a first reference point and a second reference point. Further, the first reference point is arranged at the X-ray source, and the second reference point is arranged between the X-ray source and the X-ray detector as part of the first defined arrangement.

The light guiding facility is configured to emit the light fan such that the virtual reference beam is arranged within the light fan. The control unit is configured to receive the planning information for the planned path for arranging the medical object that may be mapped using the medical X-ray device, to reposition the first defined arrangement from the initial position to the further position. The virtual reference beam intersects the planned path in the initial position and further position of the first defined arrangement. The control unit is further configured to capture the further position as the current position of the first defined arrangement. Further, the control unit is configured to rotate the second defined arrangement based on the planning information and the current position of the first defined arrangement such that the light fan illuminates the planned path.

The advantages of the X-ray device of the present embodiments substantially correspond to the advantages of the method of the present embodiments for controlling an X-ray device. Features, advantages, or alternative embodiments mentioned here may likewise also be transferred to the other claimed subject matter and vice versa.

The control unit may include an interface, a computing unit, and/or a memory unit (e.g., a memory). The control unit may be configured to control the repositioning of the first defined arrangement by providing a first control signal using the interface. Further, the control unit may be configured to control the rotation of the second defined arrangement by providing a second control signal using the interface.

In a further embodiment of the X-ray device, the light guiding facility may be arranged in the second defined arrangement on the X-ray detector, the X-ray source, or a guiding unit (e.g., a guiding device).

The light guiding facility may be attached in the second defined arrangement on the X-ray detector, the X-ray source, or the guiding unit. Alternatively or additionally, the light guiding facility may at least partially (e.g., completely) be integrated in the second defined arrangement in the X-ray detector, the X-ray source, or the guiding unit. The guiding unit may, for example, include a holder and/or a stand configured to position the light guiding facility in the second defined arrangement and to rotate the light guiding facility with the second defined arrangement.

In a further embodiment of the X-ray device, the first defined arrangement may be mounted in a translatable and/or rotatable manner.

In a further embodiment of the X-ray device, the X-ray source may be configured to emit X-rays for illuminating the medical object. The X-ray detector may be configured to capture the X-rays and to provide a signal to the control unit in dependence on the captured X-rays. The control unit may be configured to provide X-ray image data in dependence on the signal.

In a further embodiment of the X-ray device, the first and second defined arrangements may in each case be configured to be moved by a motor. Herein, the X-ray device may include a movement unit with at least one motor (e.g., an electric motor) that is configured to reposition the first defined arrangement and to rotate the second defined arrangement. The control unit may be configured to control the respective motor movement of the first and second defined arrangements. For this purpose, the control unit may provide the first and second control signals to the movement unit using the interface. The movement unit may be configured to control the first and second defined arrangement in dependence on the respective control signal.

In a third aspect, the present embodiments relate to a computer program product with a computer program that may be loaded directly into a memory of a control unit with program sections for executing all acts of a method of the present embodiments for controlling an X-ray device when the program sections are executed by the control unit.

The computer program product may, for example, include software with a source code that still has to be compiled and committed or only has to be interpreted or an executable software code that still has to be loaded into the control unit for execution. The computer program product enables the method for controlling an X-ray device by a control unit to be executed quickly, identically repeatedly, and robustly. The computer program product is configured such that the computer program product may execute the method acts according to the present embodiments using the control unit.

The computer program product is, for example, stored on a computer-readable storage medium (e.g., a non-transitory computer-readable storage medium) or held on a network or server from where the computer program product may be loaded into the processor of a control unit that may be directly connected to the control unit or may be configured as part of the control unit. Further, control information of the computer program product may be stored on an electronically readable data carrier. The control information of the electronically readable data carrier may be configured such that, when the data carrier is used in a control unit, the control information performs a method according to the present embodiments. Examples of electronically readable data carriers are DVDs, magnetic tapes, or USB sticks on which electronically readable control information (e.g., software) is stored. When this control information is read from the data carrier and stored in a control unit, all embodiments of the above-described method may be performed.

A largely software-based implementation has the advantage that it is possible to retrofit control units used to date in a simple way by a software update in order to work in the manner according to the present embodiments. In addition to the computer program, such a computer program product may optionally include additional items such as, for example, documentation and/or additional components, and hardware components such as, for example, hardware keys (e.g., dongles etc.) for using the software.

### BRIEF DESCRIPTION OF THE DRAWINGS

Example embodiments are shown in the drawings and described in more detail below. The same reference symbols are used for the same features in different figures. The figures show:

FIGS. 1 to 4 are schematic representations of various embodiments of a method for controlling an X-ray device;

FIGS. 5 to 7 are schematic representations of various embodiments of an X-ray device;

FIGS. 8 and 9 are schematic representations of various positions of the second defined arrangement; and

FIG. 10 is a schematic representation of an example embodiment of an X-ray device as a medical C-arm X-ray device.

### DETAILED DESCRIPTION

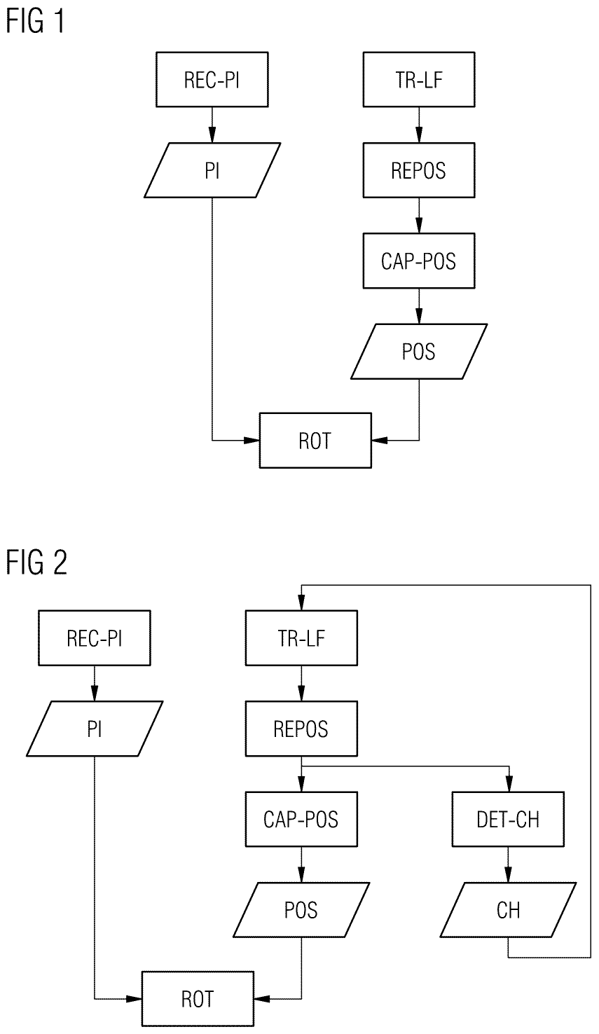

FIG. 1 shows a schematic representation of an embodiment of a method for controlling an X-ray device. Herein, the X-ray device may include an X-ray source, an X-ray detector, and a light guiding facility. Further, the X-ray source and the X-ray detector may be arranged opposite one another and in a first defined arrangement. Further, the first defined arrangement may be movably mounted. In addition, the light guiding facility may be arranged in a second defined arrangement with respect to the X-ray detector. Further, the second defined arrangement may at least be rotatably mounted. Further, a virtual reference beam may be defined by a first reference point and a second reference point. Further, the first reference point may be arranged at the X-ray source, and the second reference point may be arranged between the X-ray source and the X-ray detector as part of the first defined arrangement. In one act of the method, planning information PI may be received REC-PI for a planned path for arranging a medical object that may be mapped by the medical X-ray device. In a further act, a light fan may be emitted TR-LF by the light guiding unit such that the virtual reference beam is arranged within the light fan. In a further act, the first defined arrangement may be repositioned REPOS from an initial position to a further position. Herein, the virtual reference beam may intersect the planned path in the initial position and further position of the first defined arrangement. In a further act, the further position may be captured CAP-POS as the current position POS of the first defined arrangement. In a further act, the second defined arrangement may be rotated ROT based on the planning information PI and the current position POS of the first defined arrangement, such that the light fan illuminates the planned path.

In one embodiment, the repositioning REPOS of the first defined arrangement may be limited to rotation about an intersection point of the virtual reference beam with the planned path or a combination comprising rotation about the intersection point and translation parallel to the planned path.

In one embodiment, the first defined arrangement may be repositioned REPOS into a plurality of further positions (e.g., continuously). Herein, the virtual reference beam may intersect the planned path in the initial position and further positions of the first defined arrangement. Further, the respective current position POS of the first defined arrangement may be captured CAP-POS. In addition, the second defined arrangement may be rotated ROT based on the planning information PI and the respective current position POS of the first defined arrangement such that the light fan illuminates the planned path.

In one embodiment, the second reference point may be arranged at a center of rotation of the first defined arrangement (e.g., an isocenter) at a center of rotation of the second defined arrangement or at a geometric center of a beam exit window of the X-ray source. In addition, the virtual reference beam may be arranged along an axis of rotation of the second defined arrangement.

FIG. 2 shows a schematic representation of a further embodiment of a method for controlling an X-ray device. Herein, a change CH in a relative position of the first and second reference points may be identified DET-CH. Further, the emission of the light fan TR-LF may be adapted by the light guiding facility based on the identified change CH in the relative position.

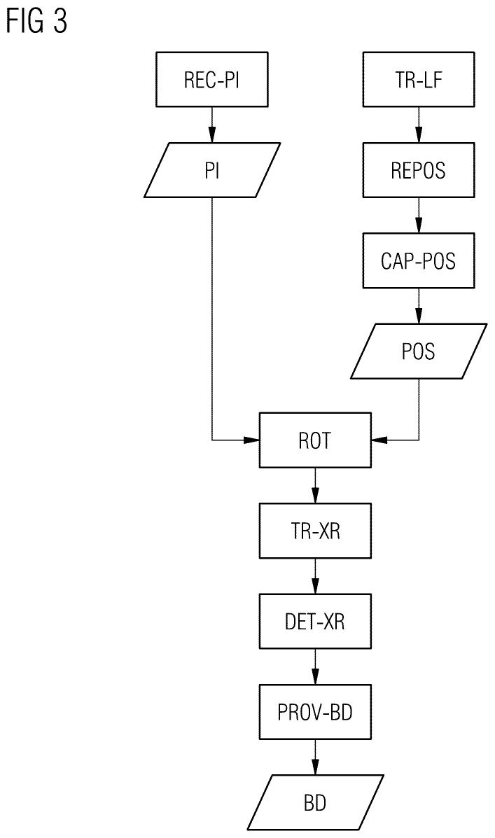

FIG. 3 shows a schematic representation of a further embodiment of a method for controlling an X-ray device. Herein, the medical object may be arranged along the planned path. Further, X-rays for illuminating the medical object may be emitted TR-XR by the X-ray source. In addition, the X-rays may be captured DET-XR by the X-ray detector, and a signal may be provided in dependence on the captured X-rays. Further, X-ray image data BD may be provided PROV-BD in dependence on the signal.

In one embodiment, the provision PROV-BD of the X-ray image data BD may include the provision of a graphical representation of the X-ray image data BD by a representation unit. Herein, the graphical representation of the X-ray image data BD may be rotated such that a current rotation of the second defined arrangement with respect to the planned path is compensated.

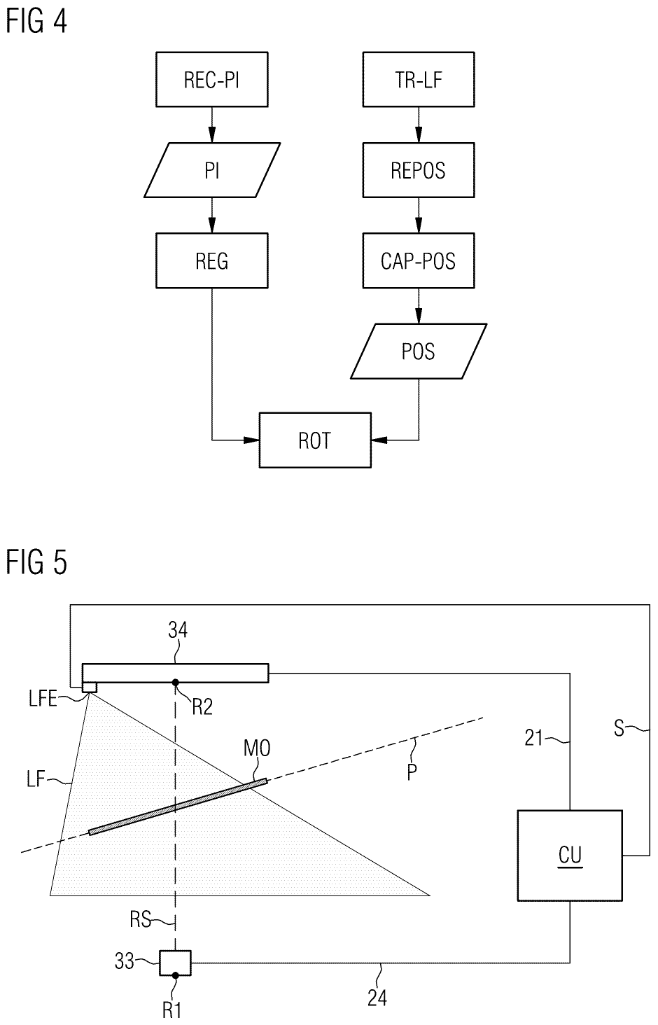

FIG. 4 shows a schematic representation of a further embodiment of a method for controlling an X-ray device. Herein, the planning information PI may be registered with a coordinate system of the X-ray device.

FIG. 5 shows a schematic representation of an embodiment of an X-ray device. The X-ray device may include an X-ray source 33, an X-ray detector 34, a light guiding facility LFE, and a control unit CU (e.g., a controller). Herein, the X-ray source 33 and the X-ray detector 34 may be arranged opposite one another and in a first defined arrangement. In one embodiment, the first defined arrangement may be movably mounted. The light guiding facility LFE may be arranged in a second defined arrangement with respect to the X-ray detector 34. Herein, the second defined arrangement may at least be rotatably mounted. Further, a virtual reference beam RS may be defined by a first reference point R1 and a second reference point R2. Herein, the first reference point R1 may be arranged at the X-ray source 33, and the second reference point R2 may be arranged between the X-ray source 33 and the X-ray detector 34 as part of the first defined arrangement. Further, the light guiding facility LFE may be configured to emit TR-LF the light fan LF such that the virtual reference beam RS is arranged within the light fan LF. The control unit CU may be configured to control the light guiding unit LFE using a signal S to emit the light fan LF. The control unit CU may be configured to receive REC-PI planning information PI for the planned path for arranging the medical object MO that may be mapped using the medical X-ray device. Further, the control unit CU may be configured to reposition REPOS the first defined arrangement from the initial position to the further position. The virtual reference beam RS may intersect the planned path P in the initial position and the further position of the first defined arrangement. Further, the control unit CU may be configured to capture CAP-POS the further position as the current position POS of the first defined arrangement. Further, the control unit CU may be configured to rotate ROT the second defined arrangement based on the planning information PI and the current position POS of the first defined arrangement such that the light fan LF illuminates the planned path P.

In one embodiment, the light guiding facility LFE may be arranged in the second defined arrangement on the X-ray detector 33. Alternatively, the light guiding facility LFE may be arranged in the second defined arrangement at the X-ray source or a guiding unit (not shown here).

In one embodiment, the first defined arrangement may be mounted in a translatable and/or rotatable manner.

FIG. 5 schematically illustrates that the second reference point R2 may be arranged at a center of rotation of the second defined arrangement (e.g., a geometric center of the X-ray detector 34, such as an X-ray sensitive surface of the X-ray detector 34).

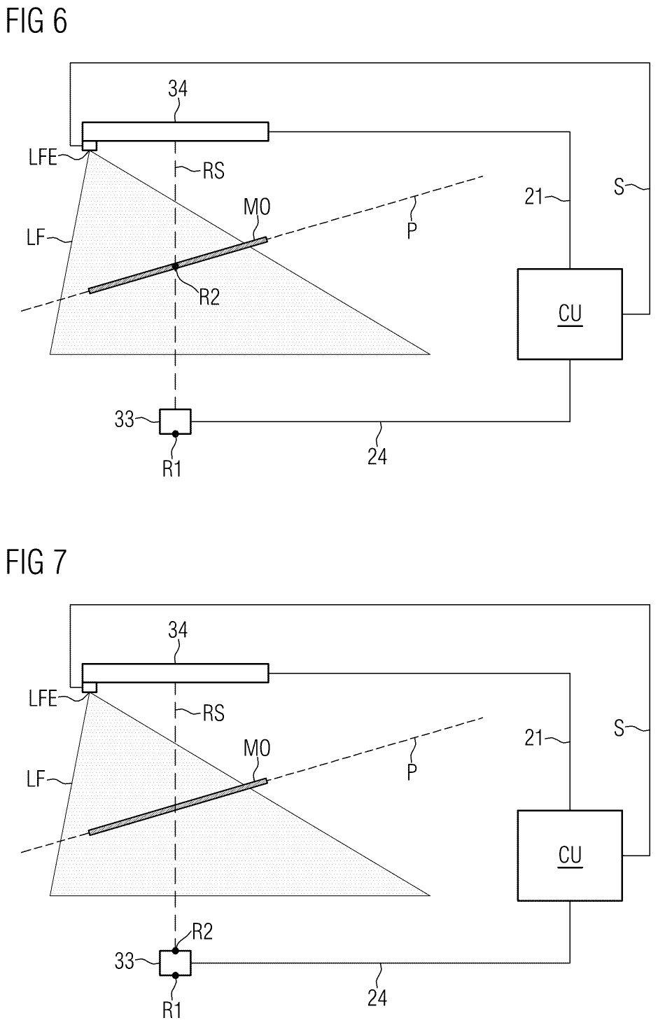

FIG. 6 shows a schematic representation of an embodiment of an X-ray device. Herein, the second reference point R2 may be arranged at a center of rotation of the first defined arrangement (e.g., an isocenter). Herein, the virtual reference beam may be arranged along an axis of rotation of the second defined arrangement.

FIG. 7 shows a schematic representation of an embodiment of an X-ray device. Herein, the second reference point R2 may be arranged at a geometric center of a beam exit window of the X-ray source 33.

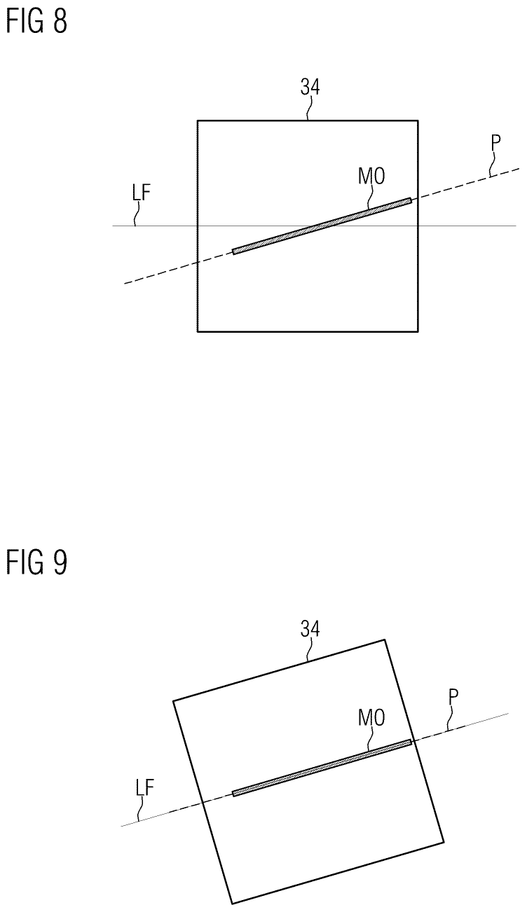

FIGS. 8 and 9 show schematic representations of various positions of the second defined arrangement. In FIG. 8, the second defined arrangement is schematically represented in a first operating state in an initial position. In the first operating state, the first defined arrangement may, for example, have been repositioned REPOS. Herein, the second defined arrangement may have an initial relative position with respect to the first defined arrangement. For example, the light fan LF may be positioned such that the light fan LF does not illuminate the planned path P in the first operating state.

In FIG. 9, the second defined arrangement is schematically represented in a second operating state in a further position. Herein, the second defined arrangement may have been rotated ROT based on the planning information PI and the current position POS of the first defined arrangement such that the light fan LF illuminates the planned path P.

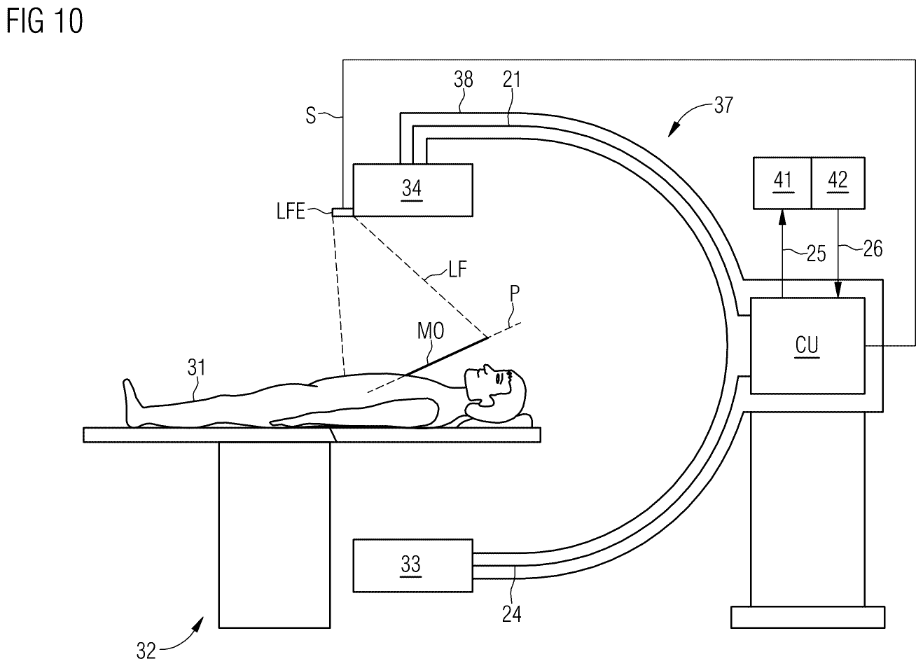

FIG. 10 shows a schematic representation of an example embodiment of an X-ray device as a medical C-arm X-ray device 37. The X-ray source 33 and the X-ray detector 34 may be arranged in a defined arrangement on a C-arm 38. The C-arm 38 may be movably mounted about one or more axes.

The control unit CU may send a signal 24 to the X-ray source 33. In response, the X-ray source 33 may emit X-rays for illuminating (e.g., for fluoroscopy of) the medical object MO and the examination object 31 positioned on a patient support apparatus 32 in dependence on the signal 24. When, after interaction with the medical object MO and the examination object 31, the X-rays strike an X-ray sensitive surface of the X-ray detector 34, the X-ray detector 34 may send a signal 21 to the control unit CU. The control unit CU may be configured to capture the X-ray image data BD based on the signal 21.

The X-ray device may further include an input unit 42 (e.g., a keyboard and/or a joystick) and a representation unit 41 (e.g., a monitor and/or a display and/or a projector). The input unit 42 may be integrated in the representation unit 41 (e.g., in the case of a capacitive and/or resistive input display). The representation unit 41 may be configured to display a graphical representation of the X-ray image data BD. For this purpose, the control unit CU may send a signal 25 to the representation unit 41. Further, the input unit may be configured to capture user input. The capturing unit 42 may further be configured to provide a signal 26 to the control unit CU in dependence on the captured user input. The control unit CU may be configured to control the X-ray device (e.g., the repositioning of the first defined arrangement) in dependence on the user input (e.g., the signal 26). For example, the repositioning of the first defined arrangement and/or the rotation of the second defined arrangement may be controlled by the input unit (e.g., a joystick or a joystick in each case). The repositioning of the first defined arrangement may be limited to rotation about an intersection point of the virtual reference beam RS and the planned path P or a combination including rotation about the intersection point and translation parallel to the planned path. This may take place by adapting the degrees of freedom of control of the input unit. Herein, the rotation of the second defined arrangement may be controlled by a joystick. Further, collimation along the planned path may be enabled. In addition, the repositioning of the first defined arrangement parallel to the planned path may be controlled by a further joystick. Herein, a center of rotation (e.g., an isocenter) of the first defined arrangement may shift along the planned path.

In one embodiment, the first and second defined arrangements may in each case be configured to be moved by a motor. Herein, the control unit CU may be configured to control the respective motor movement of the first and second defined arrangements.

The schematic representations in the described figures do not represent any scale or proportions.

Reference is made once again to the fact that the method described in detail above and the apparatuses represented are merely example embodiments that may be modified in a wide variety of ways by the person skilled in the art without departing from the scope of the invention. Further, the use of the indefinite articles “a” or “an” does not preclude the possibility that the features in question may also be present on a multiple basis. Likewise, the terms “unit” and “element” do not preclude the possibility that the components in question may consist of a plurality of interacting subcomponents, which may also be spatially distributed.

In the context of the present application, the expression “based on” may, for example, be understood in the sense of the expression “using.” For example, a formulation according to which a first feature is generated (e.g., alternatively, ascertained, determined, etc.) based on a second feature does not preclude the possibility that the first feature may be generated (e.g., alternatively, ascertained, determined, etc.) based on a third feature.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for controlling an X-ray device, wherein the X-ray device comprises an X-ray source, an X-ray detector, and a light guiding device, wherein the X-ray source and the X-ray detector are arranged opposite one another and in a first defined arrangement, wherein the first defined arrangement is movably mounted, wherein the light guiding device is arranged in a second defined arrangement with respect to the X-ray detector, wherein the second defined arrangement is at least rotatably mounted, wherein a virtual reference beam is defined by a first reference point and a second reference point, wherein the first reference point is arranged at the X-ray source, and the second reference point is arranged between the X-ray source and the X-ray detector as part of the first defined arrangement, the method comprising:
receiving planning information for a planned path for arranging a medical object that is mappable by the X-ray device;
emitting a light fan by the light guiding device, such that the virtual reference beam is arranged within the light fan;
repositioning the first defined arrangement from an initial position to a further position, wherein the virtual reference beam intersects the planned path in the initial position and the further position of the first defined arrangement;
capturing the further position as the current position of the first defined arrangement; and
rotating the second defined arrangement based on the planning information and the current position of the first defined arrangement, such that the light fan illuminates the planned path.

2. The method of claim 1, wherein the second reference point is arranged at a center of rotation of the first defined arrangement, at a center of rotation of the second defined arrangement, or at a geometric center of a beam exit window of the X-ray source.

3. The method of claim 2, wherein the center of rotation of the first defined arrangement is an isocenter of the first defined arrangement.

4. The method of claim 1, wherein the virtual reference beam is arranged along an axis of rotation of the second defined arrangement.

5. The method of claim 1, wherein the repositioning of the first defined arrangement is limited to rotation about an intersection point of the virtual reference beam with the planned path or a combination comprising rotation about the intersection point and translation parallel to the planned path.

6. The method of claim 1, further comprising identifying a change in a relative position of the first reference point and the second reference point,
wherein the emitting of the light fan is adapted by the light guiding facility based on the identified change in the relative position.

7. The method of claim 1, wherein the first defined arrangement is repositioned to a plurality of further positions,
wherein the virtual reference beam intersects the planned path in the initial position and the further positions of the first defined arrangement,
wherein the respective current position of the first defined arrangement is captured, and
wherein the second defined arrangement is rotated based on the planning information and the respective current position of the first defined arrangement, such that the light fan illuminates the planned path.

8. The method of claim 7, wherein the first defined arrangement is repositioned to the plurality of further positions continuously.

9. The method of claim 1, wherein the medical object is arranged along the planned path,
wherein the method further comprises:
emitting, by the X-ray source, X-rays for illuminating the medical object;
capturing, by the X-ray detector, the X-rays, and providing a signal depending on the captured X-rays; and
providing X-ray image data depending on the signal.

10. The method of claim 9, wherein providing the X-ray image data comprises providing, by a representation unit, a graphical representation of the X-ray image data,
wherein the graphical representation of the X-ray image data is rotated, such that the current rotation of the second defined arrangement with respect to the planned path is compensated.

11. The method of claim 1, further comprising registering the planning information with a coordinate system of the X-ray device.

12. An X-ray device comprising:
an X-ray source;
an X-ray detector;
a light guiding device; and
a controller,
wherein the X-ray source and the X-ray detector are arranged opposite one another and in a first defined arrangement,
wherein the first defined arrangement is movably mounted,
wherein the light guiding device is arranged in a second defined arrangement with respect to the X-ray detector,
wherein the second defined arrangement is at least rotatably mounted,
wherein a virtual reference beam is defined by a first reference point and a second reference point,
wherein the first reference point is arranged at the X-ray source, and the second reference point is arranged between the X-ray source and the X-ray detector as part of the first defined arrangement,
wherein the light guiding device is configured to emit the light fan, such that the virtual reference beam is arranged within the light fan,
wherein the controller is configured to:
receive the planning information for the planned path for arranging the medical object that is mappable by the X-ray device; and
reposition the first defined arrangement from the initial position to the further position, wherein the virtual reference beam intersects the planned path in the initial position and the further position of the first defined arrangement;
capture the further position as the current position of the first defined arrangement; and
rotate the second defined arrangement based on the planning information and the current position of the first defined arrangement, such that the light fan illuminates the planned path.

13. The X-ray device of claim 12, wherein the light guiding device in the second defined arrangement is arranged on the X-ray detector, the X-ray source, or a guiding unit.

14. The X-ray device of claim 12, wherein the first defined arrangement is mounted in a translatable, rotatable, or translatable and rotatable manner.

15. The X-ray device of claim 12, wherein the X-ray source is configured to emit X-rays for illuminating the medical object, and
wherein the X-ray detector is configured to capture the X-rays and to provide a signal to the controller depending on the captured X-rays, and
wherein the controller is configured to provide X-ray image data depending on the signal.

16. The X-ray device of claim 10, wherein the first defined arrangement and the second defined arrangement are in each case configured to be moved by a motor, and
wherein the controller is configured to control the respective motor movement of the first defined arrangement and the second defined arrangement.

17. In a non-transitory computer-readable storage medium that stores instructions executable by one or more processors to control an X-ray device, wherein the X-ray device comprises an X-ray source, an X-ray detector, and a light guiding device, wherein the X-ray source and the X-ray detector are arranged opposite one another and in a first defined arrangement, wherein the first defined arrangement is movably mounted, wherein the light guiding device is arranged in a second defined arrangement with respect to the X-ray detector, wherein the second defined arrangement is at least rotatably mounted, wherein a virtual reference beam is defined by a first reference point and a second reference point, wherein the first reference point is arranged at the X-ray source, and the second reference point is arranged between the X-ray source and the X-ray detector as part of the first defined arrangement, the instructions comprising:
receiving planning information for a planned path for arranging a medical object that is mappable by the X-ray device;
emitting a light fan by the light guiding device, such that the virtual reference beam is arranged within the light fan;
repositioning the first defined arrangement from an initial position to a further position, wherein the virtual reference beam intersects the planned path in the initial position and the further position of the first defined arrangement;
capturing the further position as the current position of the first defined arrangement; and
rotating the second defined arrangement based on the planning information and the current position of the first defined arrangement, such that the light fan illuminates the planned path.
